(19) United States
(12) Patent Application Publication
(10) Pub. No.: US 2022/0359071 A1
(43) Pub. Date: Nov. 10, 2022

(54) Seizure Forecasting in Wearable Device Data Using Machine Learning

(71) Applicant: Mayo Foundation for Medical Education and Research, Rochester, MN (US)

(72) Inventors: Benjamin H. Brinkmann, Byron, MN (US); Tal Pal Attia, Rochester, MN (US); Squire M. Stead, Bozeman, MT (US); Gregory A. Worrell, Rochester, MN (US); Mona Nasseri, Ponte Vedra Beach, FL (US); Boney Joseph, Rochester, MN (US); Nicholas M. Gregg, Rochester, MN (US)

(21) Appl. No.: 17/741,272

(22) Filed: May 10, 2022

Related U.S. Application Data
(60) Provisional application No. 63/186,394, filed on May 10, 2021.
(60) Provisional application No. 63/233,583, filed on Aug. 16, 2021.

(51) Int. Cl. G16H 40/67; G06N 3/04; G06N 3/08; G16H 50/20

(52) U.S. Cl. CPC G16H 40/67; G06N 3/0445; G06N 3/08; G16H 50/20

(57) ABSTRACT

Occurrence of epileptic and other seizures are predicted or otherwise forecasted in ambulatory patients using a wrist-worn device and a trained machine learning algorithm. A multi-stage training process is used to train the machine learning algorithm. A first stage of the training process is implemented on EEG data obtained from bed-ridden, or otherwise non-ambulatory, subjects. A second stage of the training process may be implemented on EEG data obtained from ambulatory subjects. A third stage of the training process is implemented on a variety of data provided by a wrist-worn device. As an example, these data can include one or more of motion data (e.g., accelerometer data), skin temperature data, heart rate data, time of day, and so on. In some implementations, training data can be taken from early portions of each patient's wearable data, while testing results can be computed from the later portions, thereby skipping transfer learning steps.

### STATEMENT REGARDING FEDERALLY SPONSORED RESEARCH

This invention was made with government support under NS073557 awarded by the National Institutes of Health. The government has certain rights in the invention.

### BACKGROUND

Despite progress in medical, surgical, and neuromodulation therapies for epilepsy, many patients continue to experience seizures. While wearable devices show promise for monitoring seizures without the expense and risks of invasive technologies, further progress is needed for widespread use of these devices. The ability to detect seizures of different semiology and forecast seizures with noninvasive sensors would be highly advantageous for establishing wearable detectors as commonly used tools in the clinical toolbox. This is, however, a challenging goal given the broad range of characteristics and lack of apparent ictal signal in non-EEG biomarkers for seizures without motor semiology.

The problem of patients self-under-reporting seizures is well established by invasive EEG devices and in-hospital EEG studies, and is of great concern in clinical epilepsy. Wearable devices may help establish objective, reliable seizure diaries for patients who are amnestic to their seizures. However, the identification of different seizure types is important to the success of wearable devices in epilepsy. To date seizure detection using non-EEG signals is challenging for non-motor seizures since the most commonly used physiological signal in seizure detection has been accelerometry.

Recent studies of seizure detection using data from wearable devices have focused on motor seizures with tonic-clonic symptoms in which the primary signal is accelerometry. Electromyography (“EMG”) has also been used for seizure detection with motor symptoms.

Other studies have used classifiers based on heart rate features. Three features can be extracted for classification, including the peak HR at the end of the HR increase, the average HR over the 60 s before the beginning of the HR increase, and the standard deviation of the HR over the 60 s before the beginning of the HR increase. The classification can be performed with a support vector machine (SVM) with a Gaussian kernel.

Advanced machine learning methods have been applied to automated seizure detection primarily focused on EEG. Recently, deep learning techniques, including convolutional neural networks (“CNN”) and recursive neural networks (“RNN”), have been investigated in EEG data to improve performance and avoid the need to identify and extract specific data features. Deep learning networks utilize vast amounts of data for training, and their training can be quite time-consuming. Transfer learning eases the hypothesis that the training data be independent and identically distributed (“i.i.d.”) with the test data. Transfer learning allows for the creation of useful classifiers with minimal training data by using preliminary training data of a different type which may be more abundant. The pre-trained model operates on low-level features, and subsequent training fine-tunes the algorithm to the target dataset. Transfer learning has been used in many applications successfully and can improve classification results, particularly when much training data is difficult to obtain.

Seizure detection from wearable devices depends heavily on the seizure type and semiology. Ambulatory training data is difficult to obtain due to the need for simultaneous gold-standard EEG confirmation of seizures. Also, data acquired during in-hospital monitoring lacks the full range of signal patterns associated with normal daily activities, especially highly active activities. Ambulatory studies with seizure diaries are possible, but self-reported diaries are notoriously inaccurate. Invasive devices capable of recording electrographic seizure activity are available for research and clinical use. They could provide objective counts of electrographic seizures, but provide limited data and cannot categorize clinical manifestations and semiology. Therefore, it is quite challenging to obtain reliable estimates of the performance and potential of seizure detection systems in real-world ambulatory use specific to seizure semiology. Such information is needed by patients, caregivers, and physicians to assess the appropriateness of wearable device systems for their particular needs. It is also advantageous to efforts to refine and optimize seizure detection systems for ambulatory use.

Furthermore, reliable seizure forecasts could potentially allow people living with recurrent seizures to modify their activities, take a fast-acting medication, or increase neuromodulation therapy to prevent or manage impending seizures. Accurate seizure forecasts have been demonstrated using invasively sampled ultralong-term EEG in ambulatory canine; however, invasive devices may not be acceptable for some patients with epilepsy, and no clinically available invasive device currently has the capability to sample and telemeter data needed for seizure forecasting. Hence, there remains a need for forecasting seizures using wearable or minimally invasive devices.

Deep learning approaches have shown promising performance for variety of difficult applications, including seizure forecasting, but many challenges exist in designing a reliable system for forecasting seizures from noninvasively recorded data. Training, testing, and validating a forecasting algorithm currently requires ultra-long duration recordings with an adequate number of seizures. Additionally, concurrent video and/or EEG validation of seizures in an ambulatory setting over months to years is logistically difficult, and is not possible using conventional in-hospital monitoring methods. Self-reported seizure diaries are the most accessible validation, but the poor reliability of such diaries is widely recognized. Performing device studies on in-hospital patients with concurrent video-EEG validation is logistically feasible, but such studies are expensive, and limited in duration, and restrict normal daily activities, which could produce false alarms, such as sports, dance, or playing a musical instrument.

### SUMMARY OF THE DISCLOSURE

The present disclosure addresses the aforementioned drawbacks by providing a method for detecting and/or forecasting a seizure in measurement data recorded with a wearable device worn by a subject. Measurement data are recorded with the wearable device, where the measurement data includes at least one of motion data, blood volume pulse data, electrodermal activity data, temperature data, or heart rate data. A trained machine learning algorithm is accessed with a computer system, where the trained machine learning algorithm has been trained on training data in order to monitor a likelihood of a seizure event occurring within signals contained in the measurement data. The measurement data are transmitted from the wearable device to the computer system. The measurement data are then applied to the trained machine learning algorithm with the computer system, generating output as an indication of at least one of detecting or forecasting a seizure event in the measurement data.

It is another aspect of the present disclosure to provide a method for training a machine learning classifier algorithm for detecting or forecasting seizure events in measurement data collected with a wearable device being worn by a subject. The method includes accessing training data with a computer system having a processor and a memory. The training data include non-ambulatory electroencephalography (EEG) data acquired from non-ambulatory subjects, ambulatory EEG data acquired from ambulatory subjects, and wearable device data acquired from subjects wearing a wearable device. An initial classifier is trained on the non-ambulatory EEG data using the computer system, generating output as a trained initial classifier. The trained initial classifier is retrained on the ambulatory EEG data using the computer system, generating output as a retrained classifier. The retrained classifier is retrained on the wearable device data with transfer learning using the computer system, generating output as a trained classifier. The trained classifier is then stored in the memory of the computer system for later use.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIGS. 1A, 1B, and 1C illustrate the architecture of an example seizure detection and/or forecasting machine learning algorithm. FIGS. 1A and 1B show a three (FIG. 1A)/four (FIG. 1B) layer LSTM network that is used to train the initial classifier (Both sections 102 and 104 are trainable) and section 104 was trained in transfer learning. FIG. 1C shows an initial classifier trained on 16 channels of iEEG data, and section 104 was retrained on wearable device data in transfer learning. Normalization and data balancing are performed on both iEEG and wearable device data and extra channels were added to the wearable device data to create 16 channels of data.

FIG. 2 is a flowchart setting forth the steps of an example method for detecting, classifying, forecasting, or otherwise monitoring seizures based on wearable device data.

FIG. 3 is a flowchart setting forth the steps of an example method for training a machine learning algorithm using a multi-stage training process in order to detect, classify, forecast, or otherwise monitor seizures based on wearable device data.

FIGS. 4A and 4B illustrate example learning schemes implemented in some embodiments described in the present disclosure. FIG. 4A shows an example transfer learning scheme: the classifier is initially trained on iEEG data from non-ambulatory iEEG data and retrained on ambulatory EEG and wearable device data and tested in leave-one-patient-out mode with the ambulatory EEG and wearable device subjects. FIG. 4B shows an example of a traditional learning scheme. The classifier is trained on the ambulatory EEG or wearable device data and tested on ambulatory EEG/wearable device data in one-patient-out/intra-subject mode.

FIG. 5 is a block diagram of an example system for detecting, classifying, forecasting, and/or monitoring seizures based on wearable device data.

FIG. 6 is a block diagram of example components that can implement the system shown in FIG. 5.

### DETAILED DESCRIPTION

Described here are systems and methods for detecting epileptic and other seizures in ambulatory patients using a wrist-worn device using a trained machine learning algorithm, such as a trained neural network. Additionally or alternatively, the onset of epileptic and other seizures in ambulatory patients can be predicted or otherwise forecasted using a wrist-worn device using a trained machine learning algorithm, such as a trained neural network.

The ability to forecast seizures minutes to hours in advance of an event has been demonstrated using invasive EEG devices, but has not been previously demonstrated using noninvasive wearable devices over long durations in an ambulatory setting. The systems and methods described in the present disclosure address and overcome limitations of previous seizure detecting and/or forecasting methods by using a multi-stage training process. As a result, the systems and methods described in the present disclosure provide for directly forecasting seizures for many patients with epilepsy without the need for an invasively implanted device.

As a non-limiting example, a first training process can be implemented on EEG data obtained from bed-ridden, or otherwise non-ambulatory, subjects. A second training process can be implemented on EEG data obtained from ambulatory subjects. As an example, these EEG data may be obtained using a portable EEG sensor. A third training process can be implemented on a variety of data provided by a wrist-worn device. As an example, these data can include one or more or motion data (e.g., accelerometer data), skin temperature data, heart rate data, and so on. The third training process may also have access to EEG data obtained from the same subjects as a gold standard. In some embodiments, the machine learning algorithm need not be re-trained on ambulatory EEG data, and thus may include the first and third training processes without the second training process described above.

Using the input data and three-stage training process, which incorporates data from both a wearable device and EEG data, the systems and methods described in the present disclosure improve upon previous methods for detecting and/or forecasting seizures. Previously, algorithms used with such wearable devices were poorly trained: they were trained either on EEG data of bed-ridden patients wearing the wrist-worn device (in which case EEG was used at the gold standard to train the algorithm, but the reduced mobility of the patients did not allow for the algorithm to be properly trained for the case of ambulatory patients), or they were trained on ambulatory patients using wrist-worn devices, but without the simultaneous availability of EEG (therefore lacking the EEG gold standard to properly train the algorithm).

Thus, in general, the systems and methods described in the present disclosure use a deep neural network approach for seizure detection and/or prediction in data obtained from a wearable device. To address the limited availability of data, the algorithm is initially trained on abundant intracranial EEG (“iEEG”). Transfer learning is then used to adapt the algorithm to biosignals from the wearable device.

It is an aspect of the present disclosure to provide a generalized deep neural network seizure detection and/or prediction algorithm trained using noninvasive signals from a first group of subjects and implemented on other subjects. As described above, it is an advantage of the present disclosure to use a three-stage learning process (e.g., a three-pass transfer learning technique) in order to overcome limited availability of training data for seizure detection and/or prediction.

In general, the machine learning algorithm can make use of a recurrent neural network (“RNN”) architecture. The RNN can include one or more long short-term memory (“LSTM”) layers, one or more convolutional layers, one or more gated recurrent unit (“GRU”) layers, and/or combinations thereof. As one non-limiting example, an algorithm with three/four layers of LSTM RNN can be designed and trained on suitable training data. For instance, the LSTM RNN can be trained on EEG data, such as 10-second iEEG segments from 2-hour recordings from subjects undergoing pre-surgical monitoring. In one example, for each subject 16 channels were selected for data extraction based on electrode placements. Channels in the brain regions involved in generating seizures can be selected. For subjects with fewer channels identifiably showing seizure activity, the adjacent channels can be added. In some implementations, the sampling frequency of the EEG data can be downsampled, such as downsampled to 400 Hz.

The algorithm can then be retrained and tested in pseudo-prospective mode on subjects with epilepsy from an implanted ambulatory device. To compensate for unbalanced ictal (or pre-ictal)/interictal data ratios in training, noise-added copies of ictal data segments, scaled by the median of each channel can be generated and used for training. The pretrained algorithm can then be adaptively retrained to detect or otherwise predict the occurrence of motor or non-motor seizures in a wearable device, such as a wrist-worn device. As an example, the wearable device may acquire one or more of accelerometer (“ACC”) data, blood volume pulse (“BVP”) data, electrodermal activity (“EDA”) data, temperature data, and heart rate (“HR”) data. In some implementations, these wearable device data can be obtained with sampling frequencies of 32 Hz, 64 Hz, 4 Hz, 4 Hz, and 1 Hz, respectively. These signals can be upsampled to a suitable sampling rate (e.g., 400 Hz) for training. The magnitude of the Fourier Transform of all signals can be calculated and added to time series data as inputs. Moreover, the signal quality indices (“SQI”) of the ACC data, BVP data, and EDA data can be measured and added as 3 additional channels. The SQI for movement in root mean square (“RMS”) accelerometry can be defined by the ratio of narrowband physiological (between 0.8-5 Hz) and broadband spectral power. The spectral entropy in the 1-3 Hz frequency band can be used to assess the signal quality of BVP. For EDA, the rate of the amplitude change in concurrent one-second windows can be calculated, resulting in 10 values for each 10-second segment.

Thus, as described, the systems and methods can implement a machine learning algorithm that is trained on initially acquired data from a patient and then using the trained algorithm to forecast all forthcoming data from that patient, with periodic retraining of the classifier. Alternatively, a machine learning algorithm can be trained using training data from many different patients and then the trained algorithm can be used to classify all forthcoming data from a particular patient, with periodic retraining of the final one or two layers of the algorithm on the patient's own data.

Accelerometry data is used to evaluate limb acceleration in 3-axes (e.g., X, Y, Z); EDA data is used to measure skin conductance, which varies with perspiration, reflecting sympathetic tone and psychological arousal; photoplethysmography (“PPG”) data can be obtained and used to evaluate microvascular blood volume changes heart rate. The wearable device data segments to retrain the classifier can be assembled into 16 channels, including ACC_{X}, ACC_{Y}, ACC_{Z}, ACC_{Mag}, BVP, EDA, TEMP, HR, FFT(ACC_{X}), FFT(ACC_{Y}), FFT(ACC_{Z}), FFT(ACC_{Mag}), FFT(BVP), FFT(TEMP), FFT(EDA), FFT(HR), SQI(ACC_{Mag}), SQI(BVP) and SQI(EDA).

10-second wearable data segments can be extracted and upsampled to 400 Hz to be adjusted to extracted iEEG segments. Each 10-second segment can be individually normalized by subtracting the average value of each channel. The whole training data including balanced ictal and interictal data, can be standardized by subtracting a population mean from an individual value and then dividing the difference by the population standard deviation. The mean and standard deviation can be used for standardizing the test data.

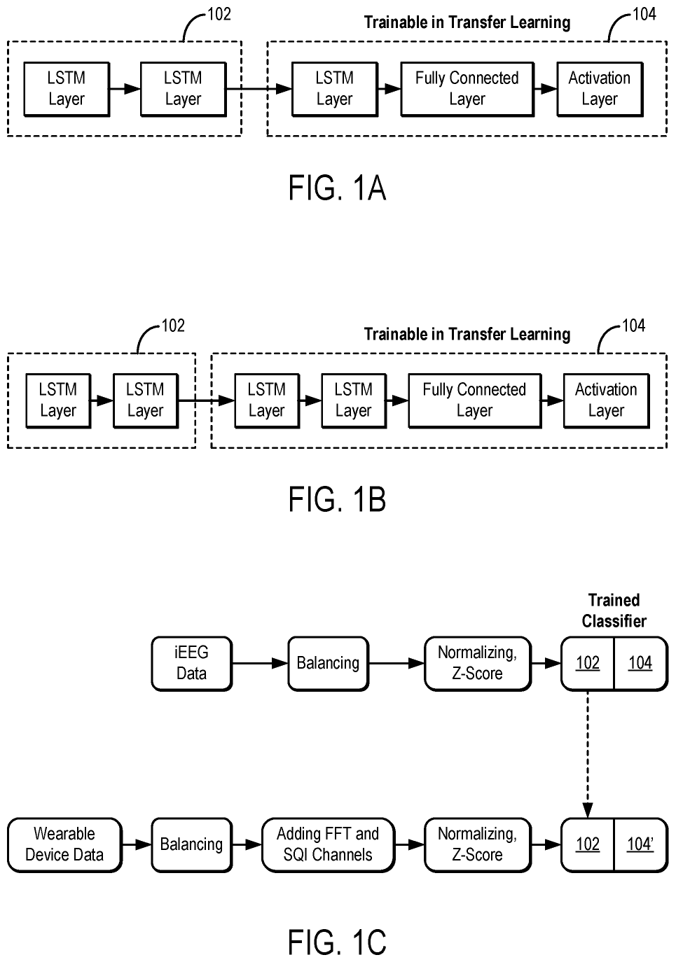

An example three-layer LSTM RNN algorithm designed as an initial classifier is shown in FIG. 1A. A unidirectional LSTM algorithm, in which the LSTM unit processes data segment time instances sequentially in one direction, is used in this example. The main component of the LSTM network is the hidden unit, which includes the memory cells. Each memory cell has three gates (forget, input, output) to control the cell behavior across time. The cell gates allow the network to detect dependencies in the stream of input data. The input to the LSTM network is in the form of a sequence input layer, and after all the LSTM layers, there is a fully connected layer and an output layer to generate the classification output using a sigmoid, or other suitable, activation function.

In one non-limiting example, three/four consecutively connected LSTM layers (with 200/128 hidden layers) and one dropout layer after each LSTM layer with a rate of 0.2 can be used. The dropout layer randomly sets input units to zero at each step during training to overcome overfitting. The network can be set to train for a selected number of epochs (e.g., 200 epochs with a batch size of 100). Additionally or alternatively, early stopping can be used to halt training when there is little to no significant improvement in the results.

The LSTM architecture can also be used for transfer learning, in which a model pre-trained on an available iEEG dataset is applied. The first two LSTM layers of the pre-trained network, noted as section 102 in FIG. 1A, can be considered non-trainable, and the rest of the network, noted as section 104 in FIG. 1A, can be set as the trainable part of the system.

In another non-limiting example, such as the one shown in FIG. 1B, four consecutively connected LSTM layers (with 128 hidden units) and one dropout layer after each LSTM layer with a rate of 0.2 can be used. The machine learning algorithm can also include a fully connected layer and an output layer to generate the classification output using an activation function, such as a sigmoid activation function. The machine learning algorithm can be trained on 60-second data segments selected from each recording. To ensure the algorithm performs seizure forecasting rather than early seizure detection, and to account for potential misalignment between the clocks in the wearable and implanted devices and the potential inexact timing of the seizure onset recorded by the device, pre-ictal data segments can be defined with a set-back of 15 minutes before the seizure onset recorded by the implanted EEG device. Lead seizures can be defined as seizures separated from preceding seizures by at least four hours, and clustered seizures can be excluded from analysis to avoid artificially inflating results.

Referring now to FIG. 1C, the iEEG data can be normalized and the ictal (or pre-ictal)/interictal training data ratio can be balanced as follows. The iEEG segments can be individually normalized by subtracting the average value of each channel following z-score normalization of the entire training data set. During training, to compensate for the unbalanced ictal (or pre-ictal)/interictal data ratio, noise-added copies of ictal (or pre-ictal) data segments can be generated. For retraining of the algorithm with data from a wearable device (e.g., ACC, BVP, EDA, HR, and TEMP data), the wearable device data can be z-score normalized and balanced as described above. The Fourier transforms of the time series signals and signal quality metrics can also be provided as channels to the LSTM algorithm.

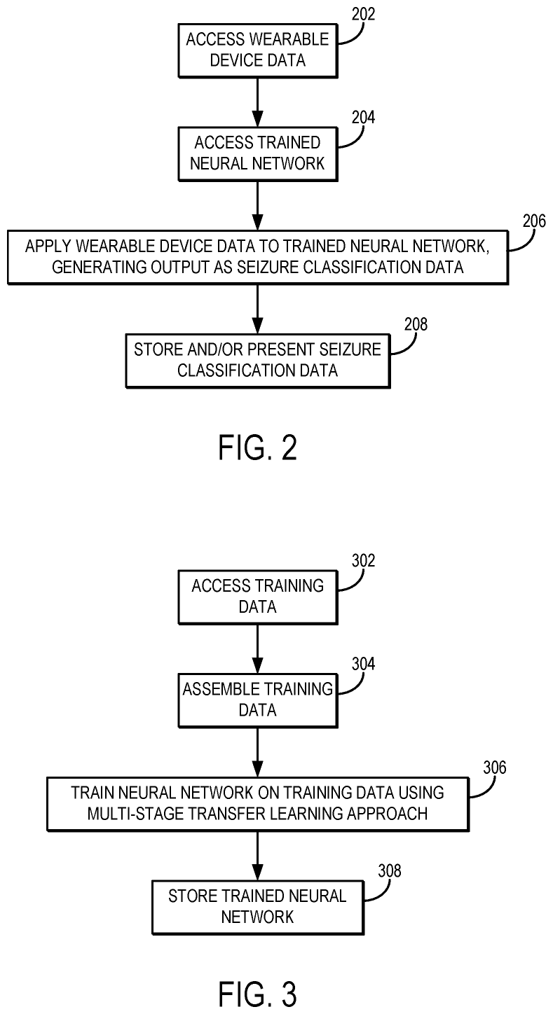

Referring now to FIG. 2, a flowchart is illustrated as setting forth the steps of an example method for detecting and/or predicting seizures using a suitably trained neural network or other machine learning algorithm. The method includes accessing wearable device data with a computer system, as indicated at step 202. Accessing the wearable device data may include retrieving such data from a memory or other suitable data storage device or medium. Alternatively, accessing the wearable device data may include acquiring such data with a wearable device and transferring or otherwise communicating the data to the computer system, which may be a part of the wearable device. As described above, wearable device data can include one or more of motion sensor data (e.g., accelerometer data), BVP data, EDA data, temperature data, and/or heart rate data. The time of day may also be recorded and used as an input to the machine learning algorithm.

A trained neural network (or other suitable machine learning algorithm) is then accessed with the computer system, as indicated at step 204. Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers or units, type of layers, ordering of layers, connections between layers, hyperparameters for layers) may be retrieved, selected, constructed, or otherwise accessed.

In general, the neural network is trained, or has been trained, on training data in order to detect and/or predict seizures based on wearable device data obtained from a subject wearing a wearable device that measures suitable signal data, such as motion data, BVP data, EDA data, temperature data, time of day, and/or heart rate data. The seizure can thus be detected and/or predicted based on the wearable device data. Additionally or alternatively, the wearable device data can be classified or otherwise characterized as being predictive of seizure onset.

The wearable device data are then input to the trained neural network, generating output as seizure classification data, as indicated at step 206. For example, the seizure classification data may include a classification of one or more of the input wearable device data as corresponding to a probable or active seizure event. For instance, the seizure classification data may indicate the probability for the subject to experience a seizure within a particular time frame, or that the subject is presently experiencing symptoms of a seizure event. As another example, the seizure classification data may indicate a classification of the input wearable device data (e.g., by assigning a particular classification to each voxel in the feature map). For instance, the trained neural network may be trained to implement automatic pattern recognition to generate seizure classification data that classify whether signals within the wearable device data correspond to, or otherwise indicate the presence of, an imminent or present seizure. Additionally or alternatively, the trained neural network may be trained to implement automatic pattern recognition to generate seizure classification data that classify whether signals within the wearable device data indicate that a seizure is likely or otherwise predicted to occur within a duration of time (e.g., within the next 60-90 minutes).

The seizure classification data generated by inputting the wearable device data to the trained neural network can then be displayed or otherwise presented to a user, stored for later use or further processing, or both, as indicated at step 208. For instance, the seizure classification data can be presented to the user as an auditory, visual, or haptic alarm indicating that a seizure event is imminent, or as such an alarm to indicate to others that the user is experiencing or about to experience a seizure. Additionally or alternatively, the seizure classification data can be presented to the user as an auditory, visual, or haptic alarm indicating that a seizure event is imminent or otherwise predicted to occur within a duration of time (e.g., within the next 60-90 minutes), or presented as such an alarm to indicate to others that the user is likely to experience a seizure within that duration of time.

Referring now to FIG. 3, a flowchart is illustrated as setting forth the steps of an example method for training one or more neural networks (or other suitable machine learning algorithms) on training data, such that the one or more neural networks are trained to receive input as wearable device data in order to generate output as seizure classification data.

In general, the neural network(s) can implement any number of different neural network architectures. For instance, the neural network(s) could implement a CNN, an RNN, an LSTM network, and so on. In some instances, the neural network(s) may implement deep learning. Alternatively, the neural network(s) could be replaced with other suitable machine learning algorithms, such as those based on supervised learning, unsupervised learning, deep learning, ensemble learning, dimensionality reduction, and so on.

The method includes accessing training data with a computer system, as indicated at step 302. Accessing the training data may include retrieving such data from a memory or other suitable data storage device or medium. Alternatively, accessing the training data may include acquiring such data with a suitable measurement system (e.g., an EEG system, a wearable device) and transferring or otherwise communicating the data to the computer system, which may be a part of the measurement system.

In general, the training data can include three sets of data: non-ambulatory EEG data, ambulatory EEG data, and wearable device data. The training data can be collected from a cohort of subjects, such as subjects who suffer from epilepsy. In some instances, the wearable device data can be collected in conjunction with non-ambulatory and/or ambulatory EEG data, which may be in addition or alternative to the other non-ambulatory EEG data and ambulatory EEG data in the training data.

Additionally or alternatively, the method can include assembling the training data from non-ambulatory EEG data, ambulatory EEG data, and wearable device data using a computer system, as indicated at step 304. This step may include assembling the non-ambulatory EEG data, ambulatory EEG data, and wearable device data into the appropriate data structure(s) on which the neural network or other machine learning algorithm can be trained.

As one non-limiting example, assembling the training data may include assembling the wearable device data into a selected data structure. For instance, as described above, the wearable device data can be processed and assembled into 16 channels of data, including ACC_{X}, ACC_{Y}, ACC_{Z}, ACC_{Mag}, BVP, EDA, TEMP, HR, FFT(ACC_{X}), FFT(ACC_{Y}), FFT(ACC_{Z}), FFT(ACC_{Mag}), FFT(BVP), FFT(TEMP), FFT(EDA), FFT(HR), SQI(ACC_{Mag}), SQI(BVP) and SQI(EDA). In other embodiments, different combinations and subsets of these data channels can alternatively be used. For instance, in some embodiments, the wearable device data can be processed and assembled into 16 channels of data, including ACC_{X}, ACC_{Y}, ACC_{Z}, ACC_{Mag}, BVP, EDA, TEMP, HR, FFT(ACC_{Mag}), FFT(BVP), FFT(TEMP), FFT(EDA), FFT(HR), SQI(ACC_{Mag}), SQI(BVP) and SQI(EDA).

As an example, training data can include ACC, BVP, EDA, TEMP, and HR signals recorded with a wearable device with sampling frequencies of 32 Hz, 64 Hz, 4 Hz, 4 Hz, and 1 Hz, respectively, which can be upsampled to 128 Hz to facilitate analysis. Signal quality metrics can be computed for ACC, BVP, and EDA and provided to the machine learning algorithm to allow the algorithm to learn and exclude poor quality data segments. The Fourier transforms of RMS accelerometry, BVP, EDA, TEMP, and HR can also be calculated and used as training data and inputs to the LSTM. Additionally, the time of day may also be used a training and input data.

Assembling the training data may include applying one or more data augmentation processes, such as generating cloned data from the non-ambulatory EEG data, ambulatory EEG data, and/or wearable device data. Cloned data can be generated by making copies of the non-ambulatory EEG data, ambulatory EEG data, and/or wearable device data while altering or modifying each copy of the respective non-ambulatory EEG data, ambulatory EEG data, and/or wearable device data. For instance, cloned data can be generated using data augmentation techniques, such as adding noise to the original data, performing a deformable transformation (e.g., translation, rotation, both) on the original data, smoothing the original data, applying a random geometric perturbation to the original data, combinations thereof, and so on. As one non-limiting example, additional training data can be generated by generating noise-added copies of ictal data segments, as described above. For instance, to compensate for the unbalanced pre-ictal/interictal data ratio in training, noise-added copies of pre-ictal data segments can be generated and used to augment the training data.

In some implementations, training data can be taken from the early part of each patient's recording, while testing results can be computed on the later portions of the patient's data. The division point between training and testing data can be chosen in each patient's recording at approximately one-third of the total record duration, and can be adjusted to ensure a minimum of four seizures for training. Consecutive 60-second data epochs can be extracted and preprocessed before being used. The training data can be normalized by subtracting its mean dividing by its standard deviation (z-scoring). The training data mean and standard deviation can be similarly used to normalize the test dataset. This setup approximates a seizure forecasting system that can be applied prospectively.

One or more neural networks (or other suitable machine learning algorithms) are trained on the training data, as indicated at step 306. In general, the neural network can be trained by optimizing network parameters (e.g., weights, biases, or both) based on the three-stage training process described above.

Training a neural network may include initializing the neural network, such as by computing, estimating, or otherwise selecting initial network parameters (e.g., weights, biases, or both). Training data can then be input to the initialized neural network, generating output data. The quality of the output data can be evaluated, such as by passing the output data to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. When the error has been minimized (e.g., by determining whether an error threshold or other stopping criterion has been satisfied), the current neural network and its associated network parameters represent the trained neural network.

In a non-limiting example, a neural network, classifier, or other machine learning algorithm can be first trained on the non-ambulatory EEG data contained in the training data. For each subject, data can be extracted from 16 channels located in areas of seizure generation. Adjacent channels can be added for subjects who have fewer than 16 channels showing seizure clearly.

In the example network described above, the last LSTM and dense layers of the initial algorithm can then be retrained using the ambulatory EEG data contained in the training data, with a leave-one-patient-out cross-validation approach to estimate performance. The effect of this retraining phase is to produce a well-tuned RNN to seizure activity. Therefore, a highly selective approach to the training data can be implemented. While cross-validation with testing on one subject and retraining performed on the other subjects can be used to estimate the algorithm accuracy and to confirm successful training, the algorithm can also be trained in pseudo-prospective mode on each subject, with the early portion of recordings used for training and subsequent data used for testing. The training/testing split can be selected such that half the recorded seizures are used for training and half for testing.

Next, the initial algorithm is retrained on the wearable device data contained in the training data. Consecutive data epochs (e.g., 10-second data epochs) can be extracted and preprocessed. The normalized training dataset, including ictal, balanced ictal, and interictal data, can be standardized by subtracting the population mean from each value and dividing by the population standard deviation. The training dataset mean and standard deviation can be used for standardizing the test dataset. Start and end times of the seizures can be determined (e.g., according to video-iEEG or scalp EEG monitoring collected in conjunction with the wearable device data), and seizures extracted from the wearable device data based on these timestamps.

Performance on subjects with motor seizures at this stage of training can be assessed using a cross-validation approach, where subjects are divided into three groups, or cross-validation folds. To assess the effectiveness of the multi-phase transfer learning approach to algorithm training, the three-fold cross-validation experiment training the algorithm can be repeated de novo at each fold, without pre-training on iEEG data.

The initial classifier can then be retrained on subjects with a range of motor and non-motor seizures, using the same cross-validation approach described previously with subjects divided into four cross-validation folds. Subjects can be chosen for each group such that the number and types of seizures in each group are as similar as possible. The cross-validation results can be stratified by seizure semiology to produce detection and/or prediction accuracy measures for each seizure type observed in the dataset.

Finally, the classifier can be re-trained on all available motor and non-motor seizures and was for ambulatory data classification.

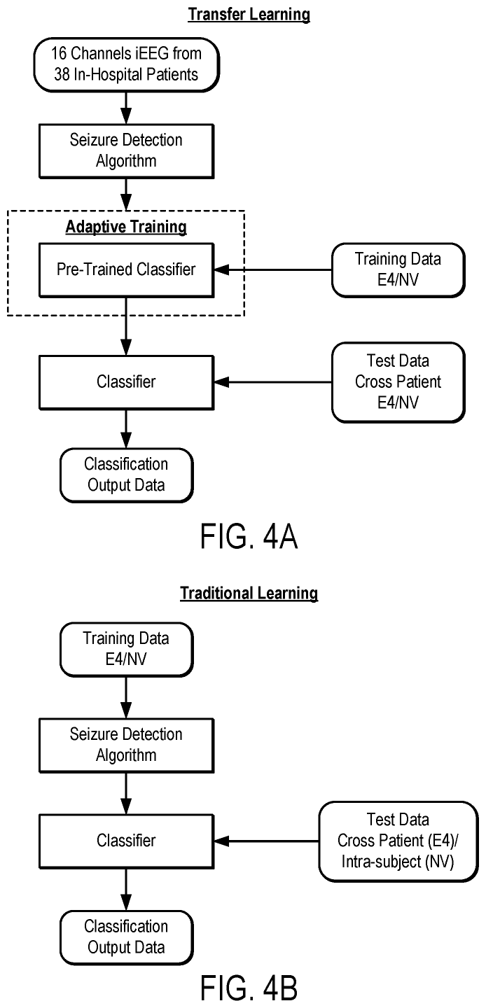

An example transfer learning scheme that can be implemented is shown in FIG. 4A and an example traditional learning scheme that can be implemented is shown in FIG. 4B.

The one or more trained neural networks are then stored for later use, as indicated at step 308. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

Described here are neural network architectures for analyzing time-series signals to detect and/or predict seizures in data recorded from noninvasive wearable devices. Transfer learning is used to adapt a classifier trained on iEEG signals to analyze wearable device data. Deep learning is a powerful technique with great potential, but some challenges have to be overcome to apply it in practice. An abundant amount of data is used to train deep learning algorithms since they learn progressively. Data availability for these algorithms for some applications may be limited, such as in epilepsy, where numbers of study subjects are limited, and independent verification of seizures is needed for reliable training and testing wearable data. Another challenge of deep learning is the massive amount of processing power typically used for training, and even with multi-core high-performance graphics processing units training can be time consuming. To overcome these challenges, the systems and methods described in the present disclosure make use of available iEEG data and use this data as an initial training set in a multi-level transfer learning approach. Using transfer learning to retrain the last two layers of the algorithm can reduce the training time by a factor of six and improve the classifier's accuracy.

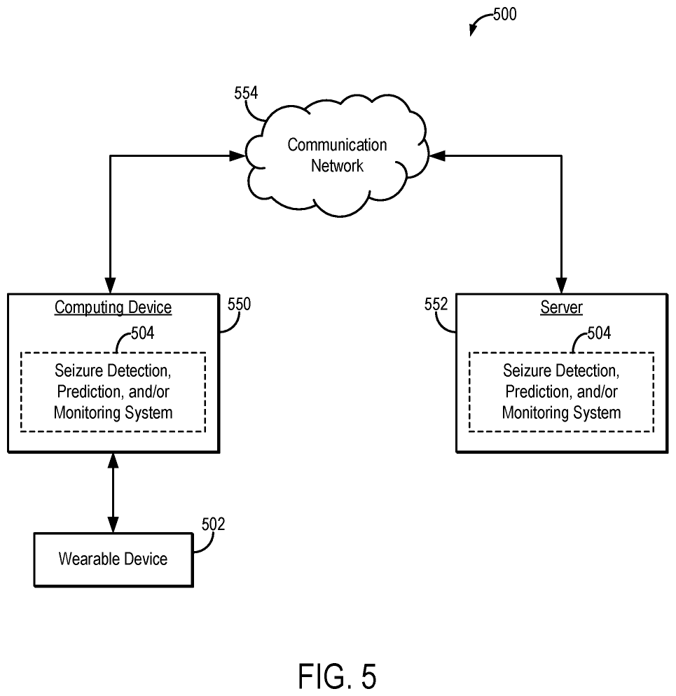

Referring now to FIG. 5, an example of a system 500 for detecting, predicting, and/or monitoring seizures based on measurement data collected by a wearable device, in accordance with some embodiments of the systems and methods described in the present disclosure is shown. As shown in FIG. 5, a computing device 550 can receive one or more types of data (e.g., motion data, BVP data, EDA data, temperature data, heart rate data) from a wearable device 502 being worn by a subject. In some embodiments, computing device 550 can execute at least a portion of a seizure detection, prediction, and/or monitoring system 504 to detect, predict, or otherwise monitor seizures from data received from the wearable device 502.

Additionally or alternatively, in some embodiments, the computing device 550 can communicate information about data received from the wearable device 502 to a server 552 over a communication network 554, which can execute at least a portion of the seizure detection, prediction, and/or monitoring system 504. In such embodiments, the server 552 can return information to the computing device 550 (and/or any other suitable computing device) indicative of an output of the seizure detection, prediction, and/or monitoring system 504.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on.

In some embodiments, wearable device 502 can be local to computing device 550. For example, wearable device 502 can be incorporated with computing device 550 (e.g., computing device 550 can be configured as part of a device for capturing, recording, and/or storing wearable device data). As another example, wearable device 502 can be connected to computing device 550 by a cable, a direct wireless link, and so on. Additionally or alternatively, in some embodiments, wearable device 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 554 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

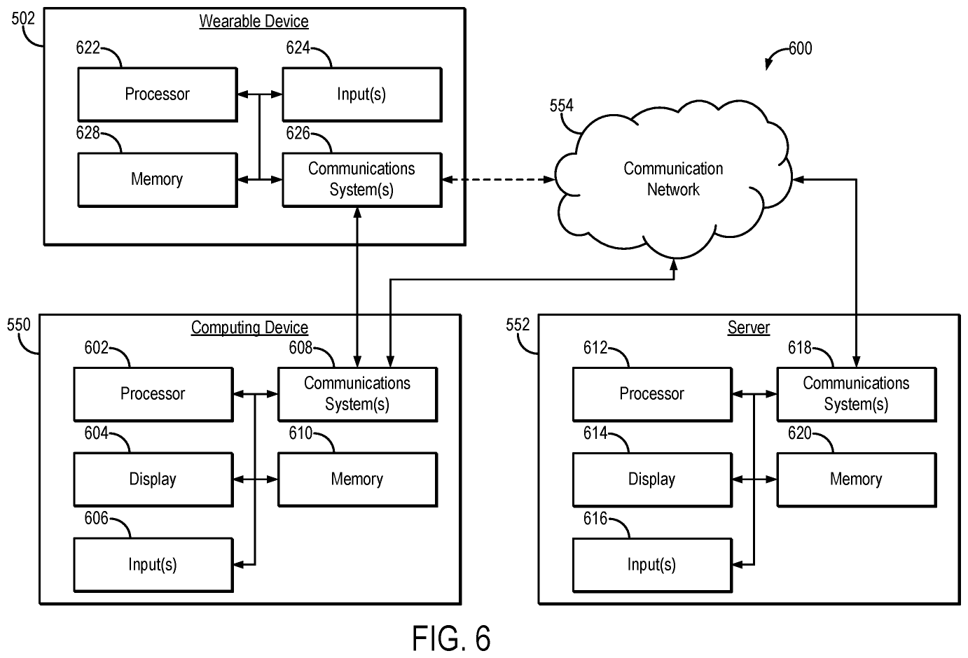

Referring now to FIG. 6, an example of hardware 600 that can be used to implement wearable device 502, computing device 550, and server 552 in accordance with some embodiments of the systems and methods described in the present disclosure is shown. As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (“CPU”), a graphics processing unit (“GPU”), and so on. In some embodiments, display 604 can include any suitable display devices, such as a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., visual alarms, user interfaces, graphics, tables), receive content from server 552, transmit information to server 552, and so on.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, a user interface) to one or more computing devices 550, receive information and/or content from one or more computing devices 550, receive instructions from one or more devices (e.g., a personal computer, a laptop computer, a tablet computer, a smartphone), and so on.

In some embodiments, wearable device 502 can include a processor 622, one or more inputs 624, one or more communications systems 626, and/or memory 628. In some embodiments, processor 622 can be any suitable hardware processor or combination of processors, such as a CPU, a GPU, and so on. In some embodiments, the one or more inputs 624 are generally configured to acquire data and can include relevant sensors or measurement devices to acquire the wearable device data described above. Additionally or alternatively, in some embodiments, one or more inputs 624 can include any suitable hardware, firmware, and/or software for coupling to and/or controlling operations of a wearable device. In some embodiments, one or more portions of the one or more inputs 624 can be removable and/or replaceable.

Note that, although not shown, wearable device 502 can include any suitable inputs and/or outputs. For example, wearable device 502 can include input devices and/or sensors that can be used to receive user input, such as a keyboard, a mouse, a touchscreen, a microphone, a trackpad, a trackball, and so on. As another example, wearable device 502 can include any suitable display devices, such as a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more inputs 624, and/or receive data from the one or more inputs 624; present content (e.g., a user interface) using a display; communicate with one or more computing devices 550; and so on. Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, and so on. In some embodiments, memory 628 can have encoded thereon, or otherwise stored therein, a program for controlling operation of wearable device 502. In such embodiments, processor 622 can execute at least a portion of the program to transmit information and/or content (e.g., data) to one or more computing devices 550, receive information and/or content from one or more computing devices 550, receive instructions from one or more devices (e.g., a personal computer, a laptop computer, a tablet computer, a smartphone, etc.), and so on.

The present disclosure has described one or more preferred embodiments, and it should be appreciated that many equivalents, alternatives, variations, and modifications, aside from those expressly stated, are possible and within the scope of the invention.

1. A method for detecting or forecasting a seizure in measurement data recorded with a wearable device worn by a subject, the method comprising:
(a) recording measurement data with the wearable device, wherein the measurement data comprise at least one of motion data, blood volume pulse data, electrodermal activity data, temperature data, heart rate data, or time of day;
(b) accessing a trained machine learning algorithm with a computer system, wherein the trained machine learning algorithm has been trained on training data in order to monitor a likelihood of a seizure event occurring within signals contained in the measurement data;
(c) transmitting the measurement data from the wearable device to the computer system; and
(d) applying the measurement data to the trained machine learning algorithm with the computer system, generating an output as an indication of at least one of detecting or forecasting a seizure event in the measurement data.

2. The method of claim 1, wherein the trained machine learning algorithm is trained on the training data using a multi-stage training process.

3. The method of claim 2, wherein the multi-stage training process includes training an initial machine learning algorithm on first training data and retraining the initial machine learning algorithm on second training data, generating an output as the trained machine learning algorithm.

4. The method of claim 3, wherein the first training data comprise non-ambulatory electroencephalography (EEG) data acquired from non-ambulatory subjects and the second training data comprise wearable device data acquired from subjects.

5. The method of claim 4, wherein the initial machine learning algorithm is retrained using transfer learning on the second training data.

6. The method of claim 5, wherein the initial machine learning algorithm is trained using a multi-layer long short-term memory (LSTM) network.

7. The method of claim 6, wherein the multi-layer LSTM network comprises at least three LSTM network layers.

8. The method of claim 6, wherein the multi-layer LSTM network comprises at least one non-trainable layer and at least one trainable layer.

9. The method of claim 8, wherein the at least one non-trainable layer comprises a first layer of the multi-layer LSTM network.

10. The method of claim 8, wherein the at least one non-trainable layer comprises two non-trainable layers and the two non-trainable layers comprise a first layer and second layer of the multi-layer LSTM network.

11. The method of claim 4, wherein the initial machine learning algorithm is first retrained on third training data comprising ambulatory EEG data acquired from ambulatory subjects before being retrained on the second training data.

12. The method of claim 2, wherein the training data comprise non-ambulatory electroencephalography (EEG) data acquired from non-ambulatory subjects, ambulatory EEG data acquired from ambulatory subjects, and wearable device data acquired from subjects.

13. The method of claim 1, wherein the measurement data comprise at least two of the motion data, the blood volume pulse data, the electrodermal activity data, the temperature data, the time of day, and the heart rate data.

14. The method of claim 1, wherein the measurement data comprise the motion data, the blood volume pulse data, the electrodermal activity data, the temperature data, time of day, and the heart rate data.

15. The method of claim 1, wherein the computer system is contained within the wearable device.

16. The method of claim 1, wherein the computer system is physically separate from the wearable device.

17. The method of claim 1, further comprising generating an alarm to a user using the wearable device when a seizure event is at least one of detected or predicted in the measurement data.

18. The method of claim 17, wherein the alarm comprises an auditory alarm.

19. The method of claim 17, wherein the alarm comprises a visual alarm.

20. The method of claim 1, wherein the output indicates that the seizure event is presently occurring within the measurement data.

21. The method of claim 1, wherein the output indicates that the seizure event is likely to occur within a duration of time.

22. The method of claim 21, wherein the duration of time is within 90 minutes.

23. The method of claim 22, wherein the duration of time is within 60 to 90 minutes.

24. A method for training a machine learning classifier algorithm for detecting or forecasting seizure events in measurement data collected with a wearable device being worn by a subject, the method comprising:
(a) accessing training data with a computer system having a processor and a memory, the training data comprising: non-ambulatory electroencephalography (EEG) data acquired from non-ambulatory subjects, ambulatory EEG data acquired from ambulatory subjects, and wearable device data acquired from subjects wearing a wearable device;
(b) training an initial classifier on the non-ambulatory EEG data using the computer system, generating output as a trained initial classifier;
(c) retraining the trained initial classifier on the ambulatory EEG data using the computer system, generating output as a retrained classifier;
(d) retraining the retrained classifier on the wearable device data with transfer learning using the computer system, generating output as a trained classifier; and
(e) storing the trained classifier in the memory of the computer system for later use.

25. The method of claim 24, wherein the subjects wearing the wearable device comprise at least one of the non-ambulatory subjects or the ambulatory subjects.

26. The method of claim 24, wherein the initial classifier is trained using a multi-layer long short-term memory (LSTM) network.

27. The method of claim 26, wherein the multi-layer LSTM network comprises at least one non-trainable layer and at least one trainable layer.

28. The method of claim 27, wherein the at least one non-trainable layer comprises a first layer of the multi-layer LSTM network.

29. The method of claim 27, wherein the at least one non-trainable layer comprises two non-trainable layers and the two non-trainable layers comprise a first layer and second layer of the multi-layer LSTM network.

30. The method of claim 24, wherein the initial classifier is first retrained on third training data comprising ambulatory EEG data acquired from ambulatory subjects before being retrained on the wearable device data.

31. The method of claim 24, wherein the wearable device data comprise at least two of subject motion data, subject blood volume pulse data, subject electrodermal activity data, subject temperature data, time of day, and subject heart rate data.
